(19) United States
(12) Patent Application Publication
(10) Pub. No.: US 2020/0371087 A1
(43) Pub. Date: Nov. 26, 2020

(54) METHODS FOR IDENTIFYING AND SEPARATING PRO-ALLERGIC SPECIFIC T CELLS

(71) Applicant: Benaroya Research Institute at Virginia Mason, Seattle, WA (US)

(72) Inventors: Eric Wambre, Seattle, WA (US); William Kwok, Bellevue, WA (US)

(21) Appl. No.: 16/635,483

(22) PCT Filed: Jul. 31, 2018
(86) PCT No.: PCT/US18/44611

§ 371 Date: Jan. 30, 2020

Related U.S. Application Data
(60) Provisional application No. 62/539,751, filed on Aug. 1, 2017.

(51) Int. Cl. G01N 33/50; C12N 5/0783; C07K 14/73; C07K 14/705; C07K 14/54

(52) U.S. Cl. CPC G01N 33/505; C12N 5/0637; C07K 2317/622; C07K 14/70578; C07K 14/54; C07K 14/70514

(57) ABSTRACT

The disclosure provides methods and compositions for labelling, detecting, quantifying, and monitoring activated allergen-specific T cells. The disclosed methods and compositions can be applied to further detect allergic states or monitor therapies for allergies in subjects in need thereof.

### CROSS-REFERENCE(S) TO RELATED APPLICATION(S)

This application claims the benefit of U.S. Patent Application No. 62/539,751, filed Aug. 1, 2017, which is incorporated herein by reference in its entirety.

### STATEMENT OF GOVERNMENT LICENSE RIGHTS

This invention was made with Government support under HHSN272200700046C and R01 AI108839 awarded by the National Institutes of Health. The Government has certain rights in the invention.

### BACKGROUND

As part of their specialization, CD4+ effector T cells acquire functional and phenotypic characteristics to specifically respond against pathogens. Within different T helper (T_{H}) cell subsets, the type 2 helper T (T_{H}2) cell subset is characterized by the production of IL-4, IL-5, IL-9 and IL-13 cytokines, which promote both IgE- and eosinophil-mediated immune responses. Although T_{H}2 cells were initially considered to be a homogeneous subset, their functional heterogeneity is now appreciated, as is the fact that additional T_{H}2 subpopulations may determine T_{H}2-driven pathology Allergen-specific T_{H}2 cells play a central role in initiating and orchestrating the allergic and asthmatic inflammatory response pathways. For example, a recent study revealed a subpopulation of human memory T_{H}2 cells that produces IL-17 along with cardinal T_{H}2 cytokines. Remarkably, the proportion of these circulating T_{H}17/T_{H}2 cells was extremely low in non-atopic individuals compared to patients with chronic severe asthma, suggesting a possible role in the pathogenesis and severity of the disease. Another source of heterogeneity among CD4+ T cell subsets is at the level of T cell-surface marker expression that determines their differentiation states, effector functions, and migratory capacity.

Allergen-specific T_{H}2 cells play a central role in initiating and orchestrating the allergic and asthmatic inflammatory response pathways. However, on major factor limiting the use of such atopic disease-causing T cells as both therapeutic targets and clinically useful biomarkers is the lack of an accepted methodology to identify and differentiate these cells from overall non-pathogenic T_{H}2 cell types. Thus, despite the advances in the art, a need remains for reliable strategies to, generate, detect, and isolate T cell subsets that are indicative of a subject's sensitivity to an allergen of interest. The present disclosure addresses this and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of specifically labeling a subpopulation of activated allergen-specific pathogenic T cells. The method comprises:

contacting a cell population comprising T cells with a suspected allergen to provide a challenged cell population,

contacting the challenged cell population with a first molecule that specifically binds to an activation marker for an activated allergen-specific T cell, wherein binding of the first molecule to the marker on a cell indicates that the cell is an activated allergen-specific T cell, and

contacting the challenged cell population, or a subpopulation thereof comprising an activated allergen-specific T cell, with a second molecule that specifically binds to a marker for a state of differentiation of the activated allergen-specific T cells.

In some embodiments, the method further comprises determining the presence of a subpopulation of activated allergen-specific T cells that is indicated to be in a terminally differentiated state.

In another aspect, the disclosure provides a method of determining whether a subject is allergic to a suspected allergen. The method comprises:

contacting whole blood or peripheral blood mononuclear cells (PBMCs) obtained from the subject with the suspected allergen to provide challenged PBMCs,

contacting the challenged PBMCs with a first molecule that specifically binds to an activation marker for an activated allergen-specific T cell, wherein binding of the first molecule to the marker on a cell indicates that cell is an activated allergen-specific T cell,

contacting the challenged PBMCs or activated allergen-specific T cells determined therefrom with a second molecule that specifically binds to a marker for a state of differentiation of activated allergen-specific T cells, and

determining the presence of a subpopulation of activated allergen-specific T cells that are indicated to be in a terminally differentiated state, wherein the presence of an activated allergen-specific T cell subpopulation that is terminally differentiated indicates that the subject is allergic to the allergen and the absence of an activated allergen-specific T cell subpopulation that is terminally differentiated indicates that the subject is not allergic to the allergen.

In some embodiments, the method further comprises treating the subject's allergic condition. In some embodiments, treating the subject comprises administering immunotherapy.

In another aspect, the disclosure provides a method of monitoring the presence of activated allergen-specific T cells in a subject allergic to the allergen. The method comprises performing the following with peripheral blood mononuclear cells (PBMCs) obtained from the subject at two or more time points:

contacting the PBMCs with the allergen to provide challenged PBMCs,

contacting the challenged PBMCs with a first molecule that specifically binds to an activation marker for an activated allergen-specific cell, wherein binding of the first molecule to the marker on a cell indicates that the cell is an activated allergen-specific T cell,

contacting the challenged PBMCs or activated allergen-specific T cells determined therefrom with a second molecule that specifically binds to a marker for a state of differentiation of activated allergen-specific T cells, and

determining the relative abundance over time of a subpopulation of activated allergen-specific T cells that are indicated to be in a terminally differentiated state, wherein a decreased abundance of terminally differentiated activated allergen-specific T cells indicates that the subject is becoming less allergic to the allergen.

In some embodiments, at least one of the two or more time points occurs during or after treatment for the subject's allergic condition. In some embodiments, the eventual absence of terminally differentiated activated allergen-specific T cells indicates that the subject is no longer allergic to the allergen.

In another aspect, the disclosure provides a method of monitoring the efficacy of the immunotherapy of a subject that is allergic to an allergen. The method comprises performing the following steps with peripheral blood mononuclear cells (PBMCs) obtained from the subject at one or more time points during immunotherapy:

contacting the PBMCs with the allergen to provide challenged PBMCs,

contacting the challenged PBMCs with a first molecule that specifically binds to an activation marker for an activated allergen-specific T cell, wherein binding of the first molecule to the marker on a cell indicates that cell is an activated allergen-specific T cell,

contacting the challenged PBMCs or activated allergen-specific T cells determined therefrom with a second molecule that specifically binds to a marker for a state of differentiation of activated allergen-specific T cells, and

determining the relative abundance over time of a subpopulation of activated allergen-specific T cells that are indicated to be in a terminally differentiated state, wherein a decreased abundance of terminally differentiated activated allergen-specific T cells indicates that the efficacy of the immunotherapy.

In another aspect, the disclosure provides a kit that comprises a first molecule that specifically binds to a marker for an activated allergen-specific T cell and a second molecule that specifically binds to a marker for a state of differentiation of activated allergen-specific T cells.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a series of illustrative scatter plots from flow cytometry analyses of activated allergen-specific T cells derived ultimately from peripheral blood mononuclear cells (PBMCs) obtained from individuals allergic and non-allergenic to peanuts.

FIG. 2 is a series of illustrative scatter plots from further flow cytometry analysis of activated allergen-specific T cells derived ultimately from peripheral blood mononuclear cells (PBMCs) obtained from allergenic individuals.

FIG. 3 is a series of illustrative scatter plots from flow cytometry analyses of activated allergen-specific T cells derived ultimately from peripheral blood mononuclear cells (PBMCs) obtained from individuals allergic and non-allergenic to peanuts.

FIG. 4 is a series of illustrative scatter plots from flow cytometry analyses of activated allergen-specific T cells derived ultimately from peripheral blood mononuclear cells (PBMCs) obtained from individuals allergic to peanuts before and after active or placebo (mock) immunotherapy involving peanut allergen exposure.

FIG. 5 graphically illustrates the different % CD27 within allergen-specific CD4+ T cells for different allergens. The allergen-specific CD4+ T cells were derived ultimately from PBMCs obtained from subjects allergic or non-allergic to the respective allergens.

FIG. 6 graphically illustrates the different % CD27 within allergen-specific CD4+ T cells for different allergens. The allergen-specific CD4+ T cells were derived ultimately from PBMCs obtained from allergic subjects before and after immunotherapy for the respective allergens.

FIG. 7 graphically illustrates the different % CD27 within allergen-specific CD4+ T cells before and after Peanut Oral Immunotherapy. The allergen-specific CD4+ T cells were derived ultimately from PBMCs obtained from allergic subjects before and after immunotherapy or placebo therapy for the allergen.

### DETAILED DESCRIPTION

The disclosure generally provides methods, and related systems and reagents, for specifically labeling, detecting, and quantifying a subpopulation of activated allergen-specific pathogenic T cells. These methods are useful for further applications, for example in determining the allergic condition of subjects to any allergen of interest, monitoring allergen-specific T cells, as well as monitoring the efficacy of immunotherapy to address allergic conditions. This disclosure is based on the inventors' characterization of a subset of human memory T_{H}2 cells confined to atopic individuals that includes all allergen-specific T_{H}2 cells. As described in more detail in Example 1, below, the inventors employed an ex vivo method using peptide-MHC Class II tetramers to detect and label activated antigen specific T cells after ex vivo exposure of collected PBMCs to the antigen of interest. The inventors determined that subsets of the antigen specific T cells were terminally differentiated CD4+ T cells as determined by further detection of CD27− and CD45RB−. These cells were also characterized by co-expression of CRT_{H}2, CD49d and CD161 and exhibit numerous functional attributes distinct from conventional T_{H}2 cells. Further analysis that these cells were involved in a distinct pathway in the initiation of pathogenic responses to allergen and elimination of these cells is indicative of clinical responses induced by immunotherapy. The inventors also employed an alternative to the ex vivo peptide-MHC Class II tetramers approach to detecting antigen specific T cells by screening instead for upregulation of CD154, which served as an activation marker for allergen specificity in the T cells. This provided a proof of concept that such an epitope-independent screen could reliably identify activated allergen specific T cells for further differentiation analysis.

The inventors expanded on these preliminary results to further develop a protocol for a dual screen employing an epitope independent activation marker for allergen specificity and a differentiation marker. As described in Example 2, the dual screen was employed to detect a subpopulation of activated allergen-specific T cells. Using CD154 upregulation as the activation marker and CD27 low (or no) expression as an indicator of terminal differentiation, the screen was able to detect activated allergen-specific T cells that are pathogenic and indicative of a subject's allergic condition and allowed meaningful monitoring of immunotherapy progress. Furthermore, it was shown that the method could be applicable to a variety of unrelated allergens of interest, demonstrating the functionality of the platform method across allergic conditions.

In accordance with the foregoing, in one aspect, the present disclosure provides a method of specifically labeling a subpopulation of activated allergen-specific pathogenic T cells. The method comprises contacting a cell population comprising T cells with a suspected allergen to provide a challenged cell population; contacting the challenged cell population with a first molecule that specifically binds to an activation marker for an allergen-specific T cell, wherein binding of the first molecule to the marker on a cell indicates that cell is an allergen-specific T cell; and contacting the challenged cell population, or a subpopulation thereof comprising an allergen-specific T cell, with a second molecule that specifically binds to a marker for a state of differentiation of allergen-specific T cells.

In certain embodiments, the method further comprises determining the presence of a subpopulation of activated allergen-specific T cells that is indicated to be in a terminally differentiated state. Indication of a terminally differentiated state can be provided by the second molecule to the marker for a state of differentiation. Depending on the specific differentiation marker, the presence or absence of the marker can be indicative of a terminally differentiated state.

Various facets of the innovation are described below under separate headings.

### Cell Population

The cell population can be any heterogeneous or homogenous population of cells that comprises or consists of T lymphocytes (“T cells”). For instance, the cell population can comprise peripheral blood mononuclear cells (PBMCs), which can be routinely isolated from blood samples obtained from a subject. In other embodiments, the cell population can be in a whole blood sample without any substantial isolation or further processing. In some embodiments, the cell population comprises T helper cells, which can typically be identified as expressing CD4 (CD4+) on their surface.

The cell population can be previously frozen or stored to facilitate convenience of performance.

In certain embodiments, the method further comprises obtaining a biological sample (e.g., whole blood) from the subject.

### Challenge with Suspected Allergen

The present disclosure addresses various methods for labelling, detecting, quantifying, and monitoring active T cells that are specific for an allergen or suspected allergen. As demonstrated in the Examples, the methods described herein are not limited by any particular allergen but can be widely applied as a platform to address any particular antigen of choice. Thus, the allergen can be a food allergen or environmental allergen. An exemplary, non-limiting list of food allergens encompassed by the present claims include peanut, soy, wheat, dairy, eggs, tree nuts (e.g., almonds, cashews, walnuts, and the like), fish (e.g., bass, cod, flounder, and the like), and shellfish (e.g., crab, lobster, shrimp, and the like). An exemplary, non-limiting list of environmental allergens include pollens (e.g., grass, tree, and the like), dust mites, pet and other animal dander, mold and mildew, and smoke and ash (e.g., from cigarettes).

The cell population can be challenged with suspected allergens in a variety of ways. The suspected allergen can be in a crude extract or isolate, such as from ground up and liquefied peanut. Alternatively, specific proteins or portions thereof can be enriched, such as being substantially or completely isolated or purified. The challenge can be quite brief to provide sufficient time for any allergen specific cells to bind to their cognate allergen epitope and be activated thereby. This can be as little as 15 minutes or so. As described in the Examples, challenges were successfully performed within an hour, but can extend for longer if necessary. Once challenged, the cell population is generally referred to as a challenged cell population. In some embodiments, the challenge occurs ex vivo. It will be apparent that in some embodiments, the challenged cell population contains cells with different developmental stages and/or expression patterns that differentiate them from the corresponding cells that previously existed in the initial pre-challenged cell population, whether as circulating in the body of the subject or in a biological sample obtained from the subject.

### Detecting Activation of Allergen-Specific T Cells

Typically, T cells specific for or activated by a particular allergen can be identified and even isolated using molecules that present the specific allergen epitopes that the T cells actually recognize. For example, ex vivo methods employing peptide-MHC class II tetramers have been used to label and enrich for T cells specific for a particular allergen. See Example 1 below, as well as, e.g., Wambre, E., et al., “Differentiation stage determines pathologic and protective allergen-specific CD4+ T-cell outcomes during specific immunotherapy,” J. Allergy Clin. Immunol. 129(2):544-551 (2012) and Wambre, E., et al., “Specific immunotherapy modifies allergen-specific CD4+ T-cell responses in an epitope-dependent manner,” J. Allergy Clin. Immunol. 133(3):872-879 (2014), each of which is incorporated herein by reference in its entirety. However, without being bound by any particular theory, using a method for allergen specificity that specifically incorporates putative epitopes of an allergen to detect specificity of cells to the allergen itself can present several drawbacks. For example, the use of the peptide-MHC class II tetramer construct to detect and isolate allergen specific T cells depends on use of the appropriate epitope, which is difficult to ensure. Furthermore, there may be multiple epitopes in any particular allergen molecule that stimulates a population of T cells and, thus, use of one or a few particular peptides conjugated with MHC class II tetramers might not capture or tag all relevant activated allergen-specific T cells. Accordingly, the epitope-based labelling of allergen specific T cells risks providing an incomplete subpopulation of activated allergen-specific T cells.

In contrast, use of a marker that does not incorporate any specific epitope molecule of the allergen to assess activation and allergen specificity would avoid such limitations. While such activation markers might be less direct (i.e., do not directly involve cell binding of the epitope itself), they can provide a more comprehensive screen of cells for activated allergen-specific cells because they depend on a binary signal of activation after ex vivo allergen challenge. Furthermore, it has been demonstrated that assays that incorporate such epitope-independent screening accurately measure activation of allergen-specific T cells and are at least equivalent to detecting allergen-specific T cells as assays based on MHC class II tetramers conjugated with the target peptide. See Renand, A., et al., “Chronic cat-allergen exposure induces a T_{H}2 cell-dependent igG4 response related to low-sensitization,” J. Allergy Clin. Immunol. 136(6):1627-1635 (2015), incorporated herein by reference in its entirety.

Accordingly, the method comprises contacting the challenged cell population with a first molecule that specifically binds to an activation marker for an allergen-specific T cell, wherein binding of the first molecule to the activation marker on a cell indicates that the cell is an allergen-specific T cell. Exemplary structures for the first molecule are described in more detail below. The term “specifically binds” refers to the ability of the first (or second) molecule to bind to the target marker, without significant binding to other molecules, under standard conditions known in the art. The first or second molecule can bind to other peptides, polypeptides, or proteins, but with lower affinity as determined by, e.g., immunoassays, BIAcore, or other assays known in the art. However, first or second molecule preferably does not cross-react with other antigens.

In certain embodiments, the method further comprises the step of determining the presence of activated allergen-specific T cells. This step comprises detecting binding of the first molecule to the activation marker for T cell allergen-specificity in one or more cells within the challenged cell population, or at least within a subpopulation thereof.

In certain embodiments, the method further comprises enriching for the activated allergen-specific T cells. This can be accomplished in various ways and typically leverages the binding of the first molecule to the activation marker. For example, the first molecule can comprise a capture domain or the like that can be bound by an immobilized or immobilizable surface, such as a bead. Such exemplary bead can be immobilized in a column or have a magnetic component to facilitate isolation of the first molecules and any activated allergen-specific T cells bound thereto.

Exemplary markers that facilitate the epitope-independent detection of activation of T cells in the challenged cell population include CD154, CD137, CD69, OX40, CD71, and CD25. For any such activation marker, the expression of the marker on the cell surface is indicative of the cells' activation (and thus, specificity to the allergen) after ex vivo challenge of the cell by the allergen. In certain embodiments, the activation marker is CD154. Methods specifically using CD154 for an activation screen of T cells are described in more detail in the Examples. In other embodiments, the activation marker is CD69.

### Marker for the State of Differentiation of Allergen-Specific T Cells

In addition to labeling and/or detecting a marker for activation of T cells, the method also comprises contacting the challenged cell population, or a subpopulation thereof comprising an allergen-specific T cell, with a second molecule that specifically binds to a marker for a state of differentiation of allergen-specific T cells. Exemplary structures for the second molecule are described in more detail below. Again, the term “specifically binds” indicates that the molecule binds to the target marker with sufficient affinity and/or avidity as to be detectable above background (i.e., to non-related structures or epitopes).

States of differentiation for allergen specific T cells can generally be categorized as terminally differentiated or not terminally differentiated. Terminally differentiated allergen specific T cells exhibit a lack of flexibility for developing or differentiating a phenotype. Prior studies have shown that terminally differentiated allergen specific T cells are associated with T_{H}2 cytokine production, which is associated with the pathogenic states of allergic responses. In contrast, the allergen specific T cells that are not yet terminally differentiated are associated with IFN-γ and IL-10 production, which can be associated with protective cells. This indicates that such non-terminally differentiated allergen specific T cells can promote tolerance to an allergen. As described in more detail in the Examples, the presence (or abundance) of terminally differentiated allergen specific T cells in a subject correlates with allergic pathologies, whereas non-terminally differentiated allergen specific T cells are found in both allergic and non-allergic (i.e., tolerant) subjects.

Markers for a state of differentiation are used to detect cells in either a terminally differentiated state or non-terminally differentiated state. In some embodiments, the method comprises detecting a state of terminal differentiation in one or more cells in the challenged cell population. Determining that one or a plurality of cells in the challenged population are in a state of terminal differentiation can comprise determining the binding status of the second molecule to the marker for the state of differentiation. In some embodiments, the method further comprises quantifying the proportion of activated allergen-specific T cells specifically bound by the second molecule to the activated allergen-specific CD4+ T cells not specifically bound by the second molecule to provide the binding status.

Markers for a state of terminal differentiation that are encompassed by this disclosure can include CD27, CD45RB, CCR7, CRT_{H}2, CCR8, CD7, CD49b, CD49d, CD161, ST2, IL17RB, HPGDS, and CD200R. Depending on the marker, terminal differentiation is detected by the expression of the marker or diminished expression of the marker, which in turn is detected by binding (or not) of the second molecule to the marker. For example, the expression of CRT_{H}2 (CRT_{H}2+), CCR8 (CCR8+), CD49b (CD49b+), CD49d (CD49d+), ST2 (ST2+), IL17RB (IL17RB+), HPGDS (HPGDS+), CD200R (CD200R+), or CD161 (CD161+) by a cell is indicative of a terminally differentiated state of the allergen-specific T cell. In this context, the expression of the marker can include normal or elevated levels of expression, detectable at the transcription or translation level. In some embodiments, the enhanced expression is detected on a population or subpopulation level, where higher numbers of cells are detected that are tagged by the second molecule, thus indicating the expression of the marker for terminal differentiation. In certain embodiments, a determined presence (or increase) of this type of marker for terminally differentiated allergen-specific T cell on one or a plurality of cells in the challenged cell population is indicative of the subject is allergic with respect to the suspected allergen.

In contrast, the diminished or lack of expression of CD27 (CD27−), CD45RB (CD45RB−), CD7 (CD7−), or CCR7 (CCR7−) by a cell is indicative of a terminally differentiated state of the allergen-specific T cell. In some embodiments, the reduced or lack of expression is detected on a population or subpopulation level, where higher numbers of cells are detected that are not tagged by the second molecule, thus indicating a plurality of cells that do not express of the marker (where absence of the marker indicates terminal differentiation). In this context, “diminished expression” can be the detection of an increased number of cells that do not express the marker for a differentiation state, thus indicating that more cells are lacking expression of the marker and, thus, are terminally differentiated. In certain embodiments, a determined lack of this type of marker for terminally differentiated allergen-specific T cell on one or a plurality of cells in the challenged cell population is indicative of the subject is allergic with respect to the suspected allergen.

In certain embodiments, the marker for a terminally-differentiated allergen-specific T cell is CD27, wherein the diminished or lack of expression of CD27 (CD27−) is indicative of a terminally differentiated state of the allergen-specific T cells.

In one embodiment, the activated allergen specific pathogenic T cell is labeled and/or detected using a first molecule that binds to CD154 and using a second molecule that binds to CD27, where cells that are CD154+ and CD27− are terminally differentiated activated allergen-specific T cells. In this embodiment, this status is pathogenic and a minimal threshold number of such cells can be indicative of an allergic state to the suspected allergen.

### First and Second Molecules

The disclosed methods incorporate use of first and second molecules to bind to activation and differentiation markers on cells in the challenged cell population. Exemplary activation and differentiation markers are discussed above. The first and second molecules are able to specifically bind to the target marker under standard conditions (see Examples below) and preferably do not bind to off-target antigens or epitopes so as to avoid providing false positive signals.

Thus, the first and second molecule can be described as affinity reagents. In certain embodiments, one or both of the first and second molecules is an antibody, or functional marker-binding fragment or derivative thereof. As used herein, the term “antibody” can be an antibody derived from any antibody-producing mammal (e.g., mouse, rat, rabbit, and primate including human), that specifically bind to the marker of interest (addressed above). Exemplary antibodies include polyclonal, monoclonal and recombinant antibodies; multispecific antibodies (e.g., bispecific antibodies); humanized antibodies; murine antibodies; chimeric, mouse-human, mouse-primate, primate-human monoclonal antibodies. The antigen-binding molecule can be any intact antibody molecule or fragment or derivative thereof (e.g., with a functional antigen-binding domain).

An antibody fragment is a portion derived from or related to a full-length antibody, preferably including the complementarity-determining regions (CDRs), antigen binding regions, or variable regions thereof. Illustrative examples of antibody fragments useful in the present disclosure include Fab, Fab′, F(ab)₂, F(ab′)₂ and Fv fragments, scFv fragments, diabodies, linear antibodies, single-chain antibody molecules, multispecific antibodies formed from antibody fragments, and the like. A “single-chain Fv” or “scFv” antibody fragment comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Antibody fragments can be produced recombinantly, or through enzymatic digestion.

Antibodies can be further modified to suit various uses. For example, a “chimeric antibody” is a recombinant protein that contains domains from different sources. For example, the variable domains and complementarity-determining regions (CDRs) can be derived from a non-human species (e.g., rodent) antibody, while the remainder of the antibody molecule is derived from a human antibody. A “humanized antibody” is a chimeric antibody that comprises a minimal sequence that conforms to specific complementarity-determining regions derived from non-human immunoglobulin that is transplanted into a human antibody framework. Humanized antibodies are typically recombinant proteins in which only the antibody complementarity-determining regions (CDRs) are of non-human origin.

Production of antibodies can be accomplished using any technique commonly known in the art. For example, the production of a polyclonal antibody can be accomplished by administering an immunogen containing the antigen of interest (i.e., the marker of interest) to an antibody-producing animal. For example, the antigen of interest (i.e., the marker of interest) can be administered to a mammal (e.g., a rat, a mouse, a rabbit, a chicken, cattle, a monkey, a pig, a horse, a sheep, a goat, a dog, a cat, a guinea pig, a hamster) or a bird (e.g., a chicken) so as to induce production of a serum containing an marker-specific polyclonal antibody. The marker of interest can be administered in combination with other components known to facilitate induction of a B-cell response, such as any appropriate adjuvant known in the art. Furthermore, the polyclonal antibody reagent can be further processed to remove or subtract any antibody members that have unacceptable affinity for antigens that are not the marker of interest. The resulting polyclonal antibody reagent will exhibit enhanced specificity for the marker of interest and are useful for detection and quantification purposes. Many approaches for adsorption of polyclonal antibody reagents to reduce cross-reactivity exist, are familiar to persons of ordinary skill in the art, and are encompassed by the present disclosure.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art.

Antibody fragments and derivatives that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art. Finally, the antibodies, or antibody fragments or derivatives can be produced recombinantly according to known techniques.

In other embodiments, one or both of the first and second molecules is an aptamer. The term “aptamer” refers to oligonucleic or peptide molecules that can bind to specific antigens of interest (i.e., marker of interest). Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phophodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer/target binding.

Peptide aptamers are protein molecules that often contain a peptide loop attached at both ends to a protamersein scaffold. The loop typically has between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display and yeast display libraries.

In some embodiments, the first or second molecule is a receptor molecule or comprises a binding domain of a receptor molecule that binds to the marker of interest. The receptor molecule can be any receptor known that can specifically bind the marker of interest as the ligand.

### Detection and Isolation

The labeling of cells in the challenged cell population permits detection of the cells and profiling of the markers expressed (or not expressed) on the cells. Detection can be performed by any known method. Typically, the first and/or second molecule comprise a detectable moiety that emits a signal under controllable circumstances. The moiety can provide, for example, a fluorescent signal upon stimulation, that permits detecting and even quantification of the signal (and thus, of binding status). Typically, the first and second molecules will have distinct detectable moieties that are mutually distinguishable. In some embodiments, the detection of binding of the first molecule to the activation marker and/or binding of the second molecule to the marker for the state of differentiation comprises use of Fluorescence-activated cell sorting (FACS) or mass cytometry (CyTOF). Where FACS allows cell sorting based on fluorescent signals emitted from fluorochromes attached to the cells via the first and/or second molecules, CyTOF utilizes heavy metal ion tags that are attached to the first and/or second molecule. Heavy metal ion tags possess much narrower signature signals in mass spectrometry to avoid signal overlap between different tagging molecules.

Optionally, the disclosed method can further comprise steps of enriching for or isolating cells that possess a particular marker profile, as determined by the binding of the first and/or second markers to the activation and/or differentiation markers, respectively. As described above, the tagging molecules (e.g., the first molecule) can comprise a domain that binds to an immobilized binding partner. The immobilized binding partner can be immobilized on a solid substrate, such as beads in a column. Alternatively, a bead solid substrate can have magnetic properties that permit immobilization of the beads and cells bound thereto via the first or second molecule and its binding partner. This allows the majority of cells not expressing the target marker (e.g., marker for activation) to be removed from cells expressing the target marker.

Alternatively, cells that are tagged (e.g., stained) with the first and/or second molecule can be selectively sorted by flow cytometry depending on the presence or absence of the first and/or second molecule. An advantage of this method is that cells determined to be positive for an activation marker, but negative for a differentiation marker (i.e., a differentiation marker where absence indicates terminal differentiation), can be detected, quantified, and even isolated.

### Further Applications

Elements of the above method can be adapted and applied for further analyses of allergen-specific T cells and potential allergic conditions.

Thus, in another aspect, the disclosure provides a method of determining whether a subject is allergic to a suspected allergen. The method comprises:

contacting whole blood or peripheral blood mononuclear cells (PBMCs) obtained from the subject with the suspected allergen to provide challenged PBMCs; contacting the challenged PBMCs with a first molecule that specifically binds to an activation marker for an allergen-specific T cell, wherein binding of the first molecule to the marker on a cell indicates that cell is an allergen-specific T cell;

contacting the challenged PBMCs or allergen-specific T cells determined therefrom with a second molecule that specifically binds to a marker for a state of differentiation of allergen-specific T cells; and

determining the presence of a subpopulation of allergen-specific T cells that are indicated to be in a terminally differentiated state, wherein the presence of an allergen-specific T cell subpopulation that is terminally differentiated indicates that the subject is allergic to the allergen and the absence of an allergen-specific T cell subpopulation that is terminally differentiated indicates that the subject is not allergic to the allergen.

In some embodiments, the presence of a minimal threshold amount of allergen-specific T cells that are terminally differentiated provides the indication of an allergic state. The threshold amount can be a ratio of activated allergen specific T cells that express the marker (+) to activated allergen specific T cells that do not express the marker (−). For example, with respect to CD27, where absence of expression indicates terminal differentiation of the activated T cell, a CD27(−):CD27(+) ratio of about 50:1, 40:1, 30:1, 20:1, 15:1, 10:1, 5:1, 1:1, 1:2, 1:5, 1:10, and the like, can serve as an indicator that sufficient terminally differentiated allergen specific T cells (i.e., pathogenic allergen-specific T cells) exist to indicate an allergy.

The method can further comprise treating the subject determined to be allergic to generate tolerance to the allergen. Such treatments can include immunotherapy, such as allergen-specific immunotherapy (ASIT; also referred to as allergen vaccine therapy). The theory of ASIT is that exposure to gradually increasing allergen exposure will decrease the population of reactive pathogenic T cells and increase the population of T cells that promote tolerance. Alternatively, the method can comprise advising the subject determined to be allergic to take precautionary measures with respect to the allergen.

In another aspect, the disclosure provides a method of monitoring the presence of allergen-specific T cells in a subject allergic to the allergen. The method comprises performing the following steps with peripheral blood mononuclear cells (PBMCs) obtained from the subject at two or more time points:

contacting the PBMCs with the allergen to provide challenged PBMCs;

contacting the challenged PBMCs with a first molecule that specifically binds to an activation marker for an allergen-specific cell, wherein binding of the first molecule to the marker on a cell indicates that the cell is an allergen-specific T cell; and

contacting the challenged PBMCs or allergen-specific T cells determined therefrom with a second molecule that specifically binds to a marker for a state of differentiation of allergen-specific T cells.

The relative abundance over time of a subpopulation of allergen-specific T cells that are indicated to be in a terminally differentiated state is determined. A decreased abundance of terminally differentiated allergen-specific T cells indicates that the subject is becoming less allergic to the allergen.

Typically, at least one of the two or more time points occurs during or after the completion of a treatment for the subject's allergic condition. Treatment can be ASIT, as described above, for the relevant allergen. In some embodiments, the steps of the method are performed a plurality of times during the course of treatment, such as 2, 3, 4, 5, 6, 7, 8, 9, 10, or more times. In some embodiments, the eventual absence (or substantial absence) of activated allergen-specific pathogenic T cells, as described herein, indicates the relative tolerance for the allergen. In some embodiments, this is indicative of a lack of allergy. The method can be performed at additional time-points thereafter as a precaution to confirm the maintenance of such tolerance.

In yet another aspect, the disclosure provides a method of monitoring the efficacy of the immunotherapy of a subject that is allergic to an allergen. The method comprises performing the following steps with peripheral blood mononuclear cells (PBMCs) obtained from the subject at one or more time points during immunotherapy:

contacting the PBMCs with the allergen to provide challenged PBMCs;

contacting the challenged PBMCs with a first molecule that specifically binds to an activation marker for an allergen-specific T cell, wherein binding of the first molecule to the marker on a cell indicates that cell is an allergen-specific T cell; and

contacting the challenged PBMCs or allergen-specific T cells determined therefrom with a second molecule that specifically binds to a marker for a state of differentiation of allergen-specific T cells.

The relative abundance over time of a subpopulation of allergen-specific T cells that are indicated to be in a terminally differentiated state is determined, wherein a decreased abundance of terminally differentiated allergen-specific T cells indicates that the efficacy of the immunotherapy.

The activation markers for an allergen-specific T cell encompassed by any of these methods are described above and are not repeated here.

In any of the described methods, the step of determining the terminal differentiated state of a subpopulation of the allergen-specific T cells comprises determining a binding status of the second molecule to the marker for a state of differentiation. The markers for a state of differentiation of the allergen-specific T cell encompassed by any of these methods are described above. As indicated above, some markers are indicative of a terminally differentiated state when positively expressed, whereas other markers (e.g., CD27) are indicative of a terminally differentiated state when they are not expressed. The status of binding (positive or negative) will indicate whether the T cell is terminally differentiated depending on the marker.

In some embodiments of any of the methods, the method further comprises quantifying the proportion of allergen-specific T cells specifically bound by the second molecule to the allergen-specific T cells not specifically bound by the second molecule to provide the binding status.

Embodiments of any of the methods can further comprise enriching for or isolating the allergen-specific T cells, as described above. Additional embodiments of the methods include the additional step of isolating the subpopulation of the allergen-specific T cells in a state of terminal differentiation. As described above, such enriching or isolating steps can comprise, for example, the use of flow cytometry or magnetic beads.

In some embodiments, any of the methods can further comprise obtaining whole blood from the subject. In some embodiments, the PBMCs can be derived from the whole blood obtained from the subject. In other embodiments, the PBMCs are within a whole blood sample without isolation therefrom.

In some embodiments of any of the methods described herein, one or both of the first and second molecules is an antibody, a marker-binding fragment or derivative thereof, an aptamer, or receptor, as described above in more detail.

### Kit

In another aspect, the disclosure provides a kit comprising a first molecule that specifically binds to a marker for an allergen-specific T cell and a second molecule that specifically binds to a marker for a state of differentiation of allergen-specific T cells. One or both of the first and second molecules can be an antibody, antibody-like molecule, receptor, aptamer, or a functional antigen-binding fragment or domain thereof. As described above in more detail, the antibody-like molecule can be a single-chain antibody, a bispecific antibody, a Fab fragment, or a F(ab)₂ fragment. The single-chain antibody can be a single chain variable fragment (scFv), single-chain Fab fragment (scFab), V_{H}H fragment, V_{NAR}, or nanobody. The first and second molecules can be configured to contain detectable moieties to provide detectable signals facilitating quantification and isolation using, for example, FACS or CyTOF.

The first and second molecules can be configured to bind to any activation and state of differentiation markers, respectively, such as the ones described above, in any combination. In a specific embodiment, the first and second molecules can be configured to bind to CD154 and CD27, respectively.

The kit can also contain various culture buffers, selected allergens or allergen extracts for challenge steps, reagents (e.g., magnetic beads) for enrichment, reagents for flow cytometry, and any other reagent that can assist the performance of any of the methods described herein, and/or written indicia for performing the methods.

Unless specifically defined herein, all terms used herein have the same meaning as they would to one skilled in the art of the present invention. Practitioners are particularly directed to Sambrook J., et al. (eds.) Molecular Cloning: A Laboratory Manual, 3rd ed., Cold Spring Harbor Press, Plainsview, N.Y. (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. Unless stated otherwise, the term “about” implies minor variation around the stated value of no more than 10% (above or below).

Disclosed are materials, compositions, and devices that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following example is provided for the purpose of illustrating, not limiting, the disclosure.

### EXAMPLES

### Example 1

### Abstract

This example describes results from a preliminary study of activated allergen-specific human T cell subpopulations that provide a signature of allergic disease or state. The initial approach included screening for activated allergen specific T cell using an epitope-MHC Class II tetramer construct to enrich and label the activated allergen specific T cells. However, in a preliminary assay an alternative approach using an activation marker, CD-154, was used.

In the study, the activated allergen specific T cells were further profiled for states of differentiation and a subset of human memory T_{H}2 cells is described that is confined to atopic individuals and includes allergen-specific T_{H}2 cells. These cells are terminally differentiated CD4+ T cells (CD27−, CD45RB−) characterized by coexpression of CRT_{H}2, CD49d and CD161 and exhibit numerous functional attributes distinct from conventional T_{H}2 cells. Transcriptome analysis further revealed a distinct pathway in the initiation of pathogenic responses to allergen and elimination of these cells is indicative of clinical responses induced by immunotherapy. Altogether, these findings identify a human T_{H}2 cell signature in allergic diseases that are useful for response monitoring and designing appropriate immunomodulatory strategies.

### Introduction

The inventors recently demonstrated that pathogenic allergen-specific T cells are highly matured effector T_{H}2 cells characterized by the lack of expression of CD27, a TNF receptor superfamily member of costimulatory molecules. Similarly, distinct subpopulations of T_{H}2 cells with enhanced function have been described in a murine model of allergic inflammation on the basis of differential expression of CXCR3 and CD62L or CCR8 and in human allergic eosinophilic inflammatory diseases according to the expression of the hematopoietic prostaglandin D synthase (hPGDS) or IL-17RB. In these studies, the researchers suggested that heterogeneity within T_{H}2-mediated immune responses plays differential roles in immunopathology. Hence, the inventors propose that allergic individuals possess specific subpopulations of T_{H}2 cells associated with global atopic inflammatory disorders.

Until now, there has been no biological measurement to accurately reflect and quantify an underlying allergic disease process, and ideally provide accurate surrogate end-points to assess immunotherapy efficacy. A major impediment to the use of allergic disease-causing T cells as a therapeutic target and clinically useful biomarker is the lack of an accepted method to both identify these cells and differentiate them from the overall T_{H}2 cell types. Recent progress in peptide-WIC class II (pMHCII) tetramer staining has allowed direct ex vivo visualization of allergen-specific CD4+ T cells and enabled quantification and characterization of these cells in a setting closer to their natural physiological state. Description of a set of T cell surface markers that are differentially expressed in allergen-specific T_{H}2 cells as compared to classical T_{H}2 cells would allow this issue to be addressed.

In this example, the inventors' preliminary work to describe an allergic T cell signature was characterized by the co-expression of the chemoattractant receptor CRT_{H}2, the natural killer cell marker CD161 and the homing receptor CD49d in human terminally differentiated (CD45RBlow CD27−) CD4+ T cells. The vast majority of allergen-specific T cells in allergic individuals with either food, pollen, pet's dander, mold or house dust mite allergy fall into this subset and were preferentially deleted during allergen-specific immunotherapy (AIT). As such, this pro-allergic subpopulation of T_{H}2 cells, confined to atopic individuals, is denoted as the T_{H}2A cell subset. Transcript analysis further highlights key functional differences between T_{H}2 cells and conventional T_{H}2 cells, providing molecular signatures that suggest specific contribution of the T_{H}2A cell subset to allergic disease. Together, these findings identify a pathogenic T_{H}2 cell signature unique to allergic individuals that could potentially be used as a clinically relevant biomarker and therapeutic target in atopic disorders.

### Results

### Allergic Disease-Related Phenotype Differences Exist in the T_{H}2 Cell Subset

For many years, chemokine receptors and surface markers have been instrumental in the characterization of memory T cell subsets with distinct migratory capacity and effector functions. To determine whether a set of T cell surface markers can be differentially expressed in allergen-specific T_{H}2 cells, a detailed ex vivo phenotypic profiling was performed of total CD4+ T cells, conventional T_{H}2 cells and allergen-specific CD4+ T cells. Using alder pollen allergy as a model, freshly isolated PBMCs from DR07:01 or DR15:01-restricted allergic individuals were stained with fluorescently-labelled pMHCII tetramers followed by magnetic column enrichment process to directly examine allergen-specific CD4+ T cell phenotypic profiles. Among T_{H}2-associated surface markers, CRT_{H}2, the prostaglandin D2 receptor chemoattractant receptor-homologous molecule expressed on T_{H}2 cells, is reported as the most reliable marker to identify human T_{H}2 cells. As a control, the ex vivo phenotypic profile of total CRT_{H}2+CD4+ memory T cells to compare with the ex vivo enriched allergen-specific CD4+ T cells. During these flow cytometric screen analyses, fluorochrome-conjugated antibodies directed against cell surface marker antigens were selected to elucidate the differentiation, maturation, activation, and homing properties of each group. As expected, ex vivo enriched allergen-specific CD4+ T cells from allergic individuals share numerous memory T_{H}2 cell features with the conventional T_{H}2 cell group featuring the expression of CD45RO, CCR4, CD200R, CD58, CD29, and CRT_{H}2. However, an allergic T cell signature was identified that includes two up-regulated (CD161 and CD49d) and four down-regulated (CD27, CD45RB, CCR7, CD7) T cell surface markers with significant differential expression (greater than 20% change, P<0.001) between groups. The CD27low, CCR7low, CD7low and CD45RBlow phenotype, which is associated with terminally differentiated memory CD4+ T cells, likely reflects recurrent natural allergen exposure. This is consistent with previous findings by our group demonstrating a strong relationship between pathogenicity of allergen-specific CD4+ T cells and the maturation stage of the cells. While loss of CD27 expression within CD4+ memory T cells is consistently associated with cells lacking CCR7 and CD7, it was observed that CD27low CD4+ T cell subset can be subdivided into two groups by CD45RB expression. Thus, in order to define a smaller set of surface markers, CD27 and CD45RB were chosen as convenient down-regulated markers reflecting allergic features.

Another striking finding from the T cell profiling was the overexpression of the C-type lectin-like receptor CD161 (4.2 fold difference, p<0.001) as part of the signature characterizing allergen-specific T_{H}2 cells. Expression of CD161 on CD4+ T cells is typically associated with T_{H}17 responses and like the conventional T_{H}2 cell subset (CRT_{H}2+ CD4+), allergen-specific T_{H}2 cells do not express the T_{H}17-associated chemokine receptor CCR6. qPCR analysis was next performed on sorted cells from allergic donors and confirmed the higher expression of CD161 mRNA in CRT_{H}2-expressing allergen-specific T cells compared to conventional T_{H}2 cells. However, while allergen-specific T_{H}2 cells express similar levels of CD161 as the T_{H}17 cell subset (CCR6+ CXCR3− CD4+), these cells did not exhibit mRNA expression of T_{H}17 phenotypic markers such as CCR6, IL23R and the transcription factor RORC. Together, these data indicate that allergic disease-related phenotypic differences (not related to a type 17 phenotype) occur in the T_{H}2 cell subset.

To demonstrate that the data were not restricted to tree pollen allergy, the ex vivo pMHCII tetramer approach was next used to characterize allergen-specific CD4+ T cells in patients with either food allergy (peanut), perennial allergy (i.e., cat and house dust mite), mold allergy (i.e., Aspergillius and Alternaria), or seasonal pollen allergy (i.e., Alder, Timothy grass). Non-allergic individuals were used as controls. Whatever the allergen tested in this study, IgE-mediated allergic diseases were characterized by high frequencies of allergen-specific CRT_{H}2+ T cells, which were strictly absent in non-allergic subjects, suggesting that presence of these CD4+ effector T cells is necessary for allergy pathogenesis. In all allergic individuals tested, the vast majority of pMHCII-tetramer positive T cells were also characterized by the lack of CD27 expression along with expression of CD161. Remarkably, CRT_{H}2+ expression on allergen-specific CD4+ T cells was concomitant with a lack of CD45RB and CD27 expression as well as co-expression of CD161 and CD49d. Collectively, these data identify the pathogenic allergen-specific T_{H}2 cell subset in atopic individuals as highly mature (CD27-CD45RBlow) T_{H}2 cells co-expressing CD161 and CD49d.

### A Distinct T_{H}2 Cell Subset is Associated with Type I Allergic Diseases

Next, the question of whether the pathogenic T cell signature identified on allergen-specific T_{H}2 cells could be used to define a subset of the T_{H}2 cells that would reflect an underlying allergic disease process was addressed. Though it has been argued that CRT_{H}2+ CD4+ T cells are present at higher frequency in allergic subjects, we observed that this difference is marginal. Despite a substantially lower proportion of CD161 expressing CRT_{H}2+ T cells in non-atopic individuals, this subset was not restricted to allergic subjects. However, it was observed that at least two markers (i.e., CD161 and CR45RB or CD27) were needed to subset the CRT_{H}2+ CD4+ T cells in order to identify an allergy-prone T_{H}2 subset virtually absent in the nonatopic group, which includes the vast majority of allergen-specific T cells from allergic individuals. A gating strategy where PBMCs were first gated according to their size, expression of CD4, and CD45RO, after the exclusion of dead cells was employed. Gates then identified CD45RBlow cells among live memory (CD45RO+) CD4+ T cells, then CD27-CD49dd+ cell subset and then CRT_{H}2+CD161+ T cell subset. It was observed that all allergic individuals tested exhibited a significantly higher number (n=80; mean±SEM, 3766±413 cells per 106 memory CD4+ T cells) of CD45^{low}CD49d+CD27− CRT_{H}2+CD161+ cells relative to non-atopic individuals (n=34, mean±SEM, 259±37 cells per 10⁶ memory CD4+ T cells, p<0.0001). As such, these pro-allergic T_{H}2 cells unique to allergic individuals were designated the T_{H}2A cell subset.

Remarkably, both conventional T_{H}2 and T_{H}2A cell subsets retain their respective phenotype after long-term clonal expansion, suggesting that they did not differ in activation or maturation status and can thus be used as a stable and relevant surrogate marker. To confirm that the T_{H}2A cell subset is specifically involved in type I allergic diseases, ten grass pollen allergic individuals were followed before and during the grass pollen season (May to August), a window of time that correlates with increased allergy symptoms and with upregulation of the activation marker CD38 within grass pollen reactive CD4+ T cells. Consistent with direct access to allergy-prone T_{H}2 cells according to CRT_{H}2, CD27, CD45RB, CD49d and CD161 differential expression, CD38 expression was observed as being specifically up-regulated within the T_{H}2A subset during grass pollen season, but not within the conventional T_{H}2 cell subset or outside pollen season. Collectively, the results demonstrate that the T_{H}2A cell subset represents a phenotypically distinct T_{H}2 subpopulation, which may encompass the vast majority of pathogenic T_{H}2 cells involved in type I allergic diseases.

### The T_{H}2A Cell Subset Represents a Suitable Therapeutic Target

To determine whether the T_{H}2A cell subset constitutes a clinically relevant therapeutic target in the allergy context, a longitudinal study was performed in a subset of peanut allergic patients completing Characterized Oral Desensitization ImmunoTherapies (CODIT) with AR101, an experimental orally administered biological drug containing the antigenic profile found in peanuts. During this randomized, double-blinded, placebo-controlled trial (ARC001), coded samples from subjects were provided to the operator at baseline both pre- and post-double blind placebo control food challenges (DBPCFC) with peanut flour, as well as at the end of the maintenance visit before DBPCFC. The magnitude and quality of peanut-specific T-cell responses were determined ex vivo using a CD154 up-regulation assay (see, Frentsch, M., et al., “Direct access to CD4+ T cells specific for defined antigens according to CD154 expression,” Nature medicine 11:1118-1124 (2005), incorporated herein by reference in its entirety) following short restimulation of PBMCs with a pool of peanut peptides library derived from Ara h 1, h 2, h 3, h 6 and Ara h 8 peanut allergic components. As expected, the vast majority of peanut-reactive CD4+ T cells were bona fide T_{H}2A cells at baseline and the DBPCFC protocol led to significant increased expression of the cell-surface activation marker CD38 concomitant with an increased average frequency of these cells. Accordingly, only T_{H}2A cells, and not conventional T_{H}2 cells, were specifically activated following peanut OFC.

As reported elsewhere, 100% and 78% of patients who completed the active treatment regimen (n=23) tolerated a cumulative amount of peanut protein of 443 mg and 1,043 mg, respectively, compared to 19% and 0% in the placebo group (n=26). In such a setting, a direct correlation was observed between decrease in peanut-specific T_{H}2A cell frequency and achievement of peanut desensitization in the active group compare to placebo. Together, these data demonstrate that T_{H}2A cells play a critical role in allergic disease pathogenesis and reinforce previous data by our group that the allergen-specific T_{H}2 cell subset may represent a suitable therapeutic target and surrogate marker of clinical efficacy during AIT.

### T_{H}2A Cells Differentially Contribute to T_{H}2-Driven Pathology

To determine whether allergic disease-related functional differences could be identified in the T_{H}2A cell subset, freshly isolated T_{H}2A, T_{H}2 (CD161−CRT_{H}2+CD27−) and T_{H}1/T_{H}17 (CD161+CRT_{H}2−CD27−) cell subsets from allergic individuals were subjected to polychromatic intracellular cytokine profile analysis. After polyclonal activation with PMA/Ionomycin, a significantly higher proportion of T_{H}2A cells expressed IL-5 and IL-9 compared to conventional T_{H}2 cells. Conversely, Interferon-Υ (IFN-Υ) and IL-17, the respective cytokines for T_{H}1 and T_{H}17 cell subsets, were restricted to the CD161+CRT_{H}2-CD27+ T helper cell population. The T_{H}2A cell subset was also more poly-functional, with a significantly greater proportion of cells producing simultaneously multiple T_{H}2 effector cytokines compared to conventional T_{H}2 cells. As a comparison, expression of cardinal T_{H}2 cytokine was also investigated within ex vivo enriched allergen-specific CD4+ T cells in allergic individuals and found to be restricted to the CD27−CRT_{H}2+CD161+ allergen-specific CD4+ T cell subset. Remarkably, the unique secretion pattern of T_{H}2A cell lines was quite stable over time, even after multiple rounds of stimulations over sequential 6-week cultures. Thus, human circulating T_{H}2A cells may contribute differently to T_{H}2-driven pathology than conventional T_{H}2 cells by simultaneously producing multiple cardinal T_{H}2 cytokines.

### Transcriptome Analysis Reveals Unique Pathway in T_{H}2A Cells

To further investigate the pathophysiologic meaning of the allergic T cell signature, a microarray analysis (GEO accession GSE93219) was performed on freshly isolated T_{H}2A cells compared to known T cell subsets (i.e., T_{H}1, T_{H}17 and T_{H}2) from different donor pools, which contained blood from 2-3 donors. This was necessary to obtain sufficient numbers of cells for microarray experiments. From the datasets comparing T_{H}2A with T_{H}2 cells, epithelium-derived cytokines receptors such as the IL-25 receptor (IL-17RB), the IL-33 receptor (IL1RL1) and the thymic stromal lymphopoietin (TSLP)-receptor (CLRF2), which are well-known molecules involved in the allergic/asthmatic immune response, were more highly expressed in T_{H}2A cells relative to conventional T_{H}2 cells. Also, it was confirmed that T_{H}2A cells produced more IL-5 and IL-9 relative to conventional T_{H}2 cells, whereas T_{H}1 and T_{H}17-related genes (IFN-g, IL-17, RORC, IL23-R and CCL20) were absent in T_{H}2 and T_{H}2A cell subset. T_{H}2A cells also highly expressed genes involved in arachidonic acid signaling that have previously been linked to allergic disease such as HPGDS, the prostaglandin synthases PTGS2, the short-chain free fatty acid receptors GPR42, and the peroxisome proliferator-activated receptors PPARG. Due to limitations of currently available anti-human ST2 and IL17RB reagents, differential expression of these two markers on the surface of peripheral CD4+ T cells was not possible using flow cytometry. Thus, to determine whether up-regulation of IL-17RB and IL1RL1 transcript identified in the T_{H}2A cell subset occurs on allergen-specific T cells from allergic individuals, a real-time PCR expression analysis was performed on sorted pMHCII tetramer-positive T cells tracking peanut-specific CD4+ T cells in peanut allergic subjects and in non-atopic individuals. Sorted conventional T_{H}2 cells from the same allergic subjects were also used as control. As expected, we confirmed that gene transcripts such as CD161, IL1RL1 and IL17RB were expressed in allergen-specific CD4+ T cells from allergic individuals, but were absent both in conventional T_{H}2 cells and in allergen-specific T cells from non-allergic individuals. These data, although not causal, imply that pathological differences between T_{H}2A and conventional T_{H}2 cells in allergic individuals are fundamental to disease development.

### Methods and Materials

### Study Design

The main research objective of this study was to determine whether allergic individuals possess specific subpopulations of T_{H}2 cells associated with global atopic inflammatory disorders. To investigate allergic-related differences in peripheral T cells from allergic individuals, the profile of allergen-specific T_{H}2 cell subset was determined ex vivo using direct pMHCII tetramer staining, and compared to the profile of total T_{H}2 cell subset. Candidate signature associated markers were then tested in allergic patients and in non-atopic individuals. To evaluate this signature in the context of clinical intervention, a longitudinal study was conducted in patients receiving Oral Immunotherapy. Sample size was determined on the basis of the availability of fresh blood samples and with the intention to include samples before and after oral food challenge, and before and after therapy, where possible. All data generated were included in the analysis. Researchers performing the measurements were blinded to the treatment group and sample identity. To further explore the pathophysiologic meaning of this allergic T cell signature, real-time PCR, intracellular cytokine analysis and microarray analysis were used.

### Subjects

Subjects were recruited at the Allergy Clinic at Virginia Mason Medical Center (Seattle, Wash.). All subjects were recruited with informed consent and the study was approved by the Institutional Review Board of Benaroya Research Institute (Seattle, Wash.). Allergic subjects (n=80) were selected based on their clinical history, a positive prick test and positive IgE reactivity to extract (test score ≥0.35 kU/L) using the ImmunoCap test (Phadia AB). For subjects with no history of allergy (n=34), the non-atopic status was confirmed by a lack of IgE reactivity and a negative in vitro basophil activation assay following stimulation with a pool of allergen extracts. All subjects were HLA typed by using sequence specific oligonucleotide primers with Unitray SSP Kits (Invitrogen).

### CODIT Study Design and Participants

In ARC001, a multicenter, randomized, double-blind placebo-controlled study of efficacy and safety of characterized oral desensitization immunotherapy (CODIT™) (Aimmune Therapeutics Inc), peanut allergic subjects aged 4 to 26 years were enrolled based on clinical history of allergy to peanut; a serum IgE to peanut of ≥0.35 kUA/L (UniCAP) or positive skin prick test to peanut >3 mm compared to control; and an allergic reaction at or before 100 mg of peanut protein during a screening double-blind placebo-controlled food challenge (DBPCFC), conducted in accordance with PRACTALL (Practical Issues in Allergology, Joint United States/European Union Initiative) guidelines. Participants were randomly assigned (1:1) to active treatment with AR101 or matched placebo. Subjects initiated the study with a single dose of 0.5 mg of study product and escalated biweekly over the course of approximately 20 weeks to the target maintenance dose of 300 mg/day. The primary clinical efficacy endpoint was the proportion of subjects in each group who tolerated at least 300 mg (443 mg cumulative) of peanut protein with no more than mild symptoms at the Exit DBPCFC. Out of 55 subjects enrolled in the ARC001 study, 10 participants were consented for additional volume of blood (10 to 15 mL) to be collected before and after the screening DBPCFC, and 7 participants (3 placebo and 4 active) were consented for additional volume of blood to be collected pre- and post-CODIT.

### Tetramer Reagents

Biotinylated HLA-DR molecules were generated and purified as described. T cell epitopes were identified by Tetramer Guided Epitope Mapping. Epitope specific pMHCII tetramer reagents were generated by loading specific peptides onto biotinylated soluble DR monomers, and subsequently conjugated with PE-streptavidin.

### Ex Vivo Analysis of Allergen-Specific CD4+ T Cells

Twenty million PBMCs in culture medium at a concentration of 150 million cells/ml were treated with dasatinib (49) for 10 min at 37° C. followed by staining with 20 μg/ml PE-labeled pMHCII tetramers at room temperature for 100 min. After tetramer staining, cells were then washed twice and incubated with anti-PE magnetic beads (Miltenyi Biotec, Bergisch Gladbach, Germany) at 4° C. for another 20 min. The cells were washed again and enriched using a magnetic column according to the manufacturer's instructions (Miltenyi Biotec, Auburn, Calif.). Frequency was calculated as previously described (50). For unbiased-FACS screen analysis, CRT_{H}2-labelled PBMCs and cells in the tetramer bound fractions were both stained with antibodies against markers of interest or corresponding isotypematched mAbs. A combination of the vital dye ViaProbe (BD PharMingen) as a viability marker, CD19 (ebiosciences) and CD14 (ebiosciences) was used to exclude dead cells, B cells and monocytes from the analysis, respectively. A FACS AriaII was used for multi-parameter analysis, and data were analyzed with FlowJo software (Treestar, Ashland, Oreg).

### T_{H}2A Cell Subset Analysis

T_{H}2A cells were defined as CD4+CD45RO+CD27−CD45RB^{low}CRTH2+CD161+CD49d+ T cell subset. The following antibodies were used in flow cytometry analysis: FITC-conjugated anti-CD45RB (clone MEM-55) from AbD Serotec, ECD-conjugated anti-CD45RO (clone UCHL1) from Beckman Coulter, Alexa Fluor 647-conjugated anti-CRT_{H}2 (clone BM16), APC-H7-conjugated anti-CD27 (clone M-T271), all from BD Biosciences, V450-conjugated anti-CD38 (clone HIT2), eFluor650-conjugated anti-CD3 (clone OKT3), PE-conjugated anti-CD161 (clone HP-3G10), all from eBiosciences and PE-Cy7-conjugated anti-CD49d (clone 9F10), BV605-conjugated anti-CD4 (clone OKT4), all from Biolegend. CD45RBlow cells were identified using a cutoff of 35% among live memory CD4+ T cells.

### T_{H} Cell Subset Isolation

Freshly isolated PBMCs were labeled with V500-conjugated anti-CD4 (clone RPA-T4), AF647-conjugated anti-CRT_{H}2 (clone BM16), PE-Cy7-conjugated anti-CCR6 (clone R6H1), AF488-conjugated anti-CXCR3 (clone 1C6/CXCR3), APC-H7-conjugated CD27 (Clone M-T271) all from BD Biosciences, ECD-conjugated anti-CD45RO (clone UCHL1; Beckman Coulter), PE-conjugated anti-CD161 (clone HP-3G10) and eFluor 650-conjugated anti-CD3 (clone OKT3) from eBiosciences. A combination of the vital dye ViaProbe (BD PharMingen) as a viability marker, CD19 (eBiosciences) and CD14 (eBiosciences) was used to exclude dead cells, B cells and monocytes from the analysis, respectively. T_{H}2A cells (CD4+CD45RO+CD27−CRT_{H}2+CD161+), conventional T_{H}2 cells (CD4+CD45RO+CD27−CRT_{H}2+CD161−), T_{H}17 cells subset (CD4+CD45RO+CRT_{H}2−CCR6+CXCR3−) and T_{H}1 cells (CD4+CD45RO+CRT_{H}2−CCR6−CXCR3+) were isolated to a purity over 96% using FACS ARIAII (BD Biosciences).

### Intracellular Cytokine Staining

Intracellular staining was performed by using the Cytofix/Cytoperm buffer set (BD Biosciences) according to the manufacturer's instructions. Briefly, cells were incubated for 5 hours at 37° C., 5% CO₂ with PMA (50 ng/ml), ionomycin (500 ng/ml) and GolgiPlug (BD Biosciences, CA), then permeabilized with Cytofix/Cytoperm buffer and stained with APC-conjugated anti-IL-5 (JES1-39D10) from Miltenyi Biotec, FITC-conjugated anti-IL-4 (clone 8D4-8) from eBiosciences and PEconjugated anti-IL-9 (clone MH9A4), PerCP/Cy5.5-conjugated anti-IL-13 (clone JES10-5A2), BV510-conjugated anti-IFN-g (clone 4S.B3), APC/Cy7-conjugated anti-IL-17 (clone BL168) all from Biolegend. After 30 min at 4° C., cells were washed and immediately analyzed by flow cytometry.

### Real-Time PCR Expression Analysis

The Fluidigm BioMark 96.96 Dynamic Array (51) was used to measure the gene expression in small cell populations. Ten cells per well were sorted by FACS in quadruplicate into 96-well plates containing a reaction mix for reverse transcription (CellsDirect One-Step qRT-PCR kit; Invitrogen) and pre-amplification with 96 selected gene primer pairs (DELTAgene assays, Fluidigm). After sorting, samples were reverse transcribed and pre-amplified for 18 cycles. Primers and dNTPs were removed by incubation with ExonucleaseI (NE Biolabs), and samples were diluted (5×) with TE buffer and stored at −20° C. Samples and assays (primer pairs) were prepared for loading onto 96.96 Fluidigm Dynamic arrays according to the manufacturer's recommendations. Briefly, the sample was mixed with 20× DNA Binding Dye Sample Loading Reagent (Fluidigm Corp.), 2× SsoFast EvaGreen Supermix with Low ROX (BioRad). Assays were mixed with 2× assay loading reagent (Fluidigm Corp.) and TE to a final concentration of 5 μM. The 96.96 Fluidigm Dynamic Arrays (Fluidigm Corp.) were primed and loaded on an IFC Controller HX (Fluidigm Corp.) and real-time PCR was run on a BiomarkHD (Fluidigm Corp.). Data were collected and analyzed using Fluidigm Real-Time PCR Analysis software (v4.1.2).

### Microarray Analysis and Data Analysis

Conventional T_{H}1 cells, conventional T_{H}17 cells, T_{H}2A cells and conventional T_{H}2 cells were sorted from PBMCs of allergic subjects, as described above. Use of donor pools (each pool containing blood from 2-3 donors) was necessary to obtain sufficient numbers of cells for microarray experiments. Sorted T_{H} subsets were stimulated for 6 hours with anti-CD3/CD28 beads (Life Technologies) or left unstimulated prior to extraction of RNA (RNeasy Mini kit; Qiagen). Replicates of RNA were obtained from each sample that passed quality control. cRNA was prepared by amplification and labelling using the Illumina TotalPrep RNA Amplification Kid (Life Technologies) and hybridized to Human HT-12 Beadarray Chips (Illumina). Beadchips were scanned on a HiScanSQ (Illumina). Background subtracted data was generated using GenomeStudio Software (Illumina). Data were processed by customized R/bioconductor pipeline, including quantile normalization, flooring, log2 transformation and PALO filtering (Present At Least Once; at least one sample must have had detection p-value <0.01). Analyses was performed using R.

### Statistical Analysis

Prism software (GraphPad) was used for statistical analysis of flow cytometry data. No randomization or exclusion of data points was used. The nonparametric Mann-Whitney U test was used for unpaired comparisons between groups, whereas the nonparametric Wilcoxon matched pairs test was used for paired comparison.

### Conclusion

Although antigen-specific T_{H}2 cells are at the core of the allergic process in atopic individuals, tracking and targeting these allergic disease-causing T cells without affecting other non-pathogenic T_{H}2 processes has been a challenge. In this study, an ex vivo pMHCII tetramer-based T cell profiling was conducted, leading to the demonstration that in all type 1 allergic individuals, the differential expression of at least three markers (i.e., CRT_{H}2, CD161 as well as a differentiation stage marker such as CR45RB or CD27) define a pathogenic T_{H}2 cell subset that is allergen-specific and virtually absent in non-atopic individuals (denoted here as T_{H}2A subset). Multiples lines of evidence suggested the pathogenic potential of T_{H}2A cell subset in settings of allergic inflammatory disease. First, it was observed that allergen-specific T_{H}2 cells from allergic patients with either seasonal, perennial, fungus or food allergy were virtually all contained in the terminally differentiated (CD27−) memory T helper cell subset. These typically co-expressed CRT_{H}2 and CD161. Second, the overall number of cells from this subset was dramatically higher in all allergic individuals as compared to non-atopic individuals. This particular proallergic T helper cell subset is remarkable in that it can easily be detected directly ex vivo in every allergic individual due to its ability to include a broad array of allergen-specific T_{H}2 cells. As such, these data demonstrate that during a natural allergen challenge, such as pollen season or a peanut challenge test, the T_{H}2A cell subset is distinctively activated indicating that the T_{H}2A cells are important in the pathogenesis of allergic diseases. Finally, the data highlight key functional and molecular differences between pathogenic and conventional T_{H}2 cells, recapitulating previous observation in their murine counterpart and highlighting specific therapeutic targets. Further detailed studies focusing on the T_{H}2A cell subset may prove useful in the diagnosis, molecular characterization or the discovery of novel therapeutic targets to enhance the power of allergen vaccines.

It is notable that this study included a follow-on analysis of activated allergen-specific T_{H}2 cells that incorporated an alternative approach to profile the activated allergen-specific T_{H}2 cells. Instead of using an epitope based screen to establish allergen specificity and activation, upregulation of an activation marker, CD154, was used as a screening target. This facilitated the ex vivo analysis and profiling of T_{H}2 cells from PBMCs obtained from subjects participating in a longitudinal study of allergic subjects receiving peanut allergen exposure. The CD154 upregulation screen was able to identify the population of activated allergen-specific T_{H}2 cells after ex vivo allergen challenge. The challenged allergen-specific T_{H}2 cells were then profiled for differentiation markers and were determined to be identifiable as terminally differentiated with negative or low CD27 expression. This epitope-independent double screen is explored further below in Example 2.

### Example 2

### Abstract

This example describes methods of detecting, separating, and/or monitoring the presence of pro-allergic T cells to a particular allergen of interest in a subject. The exemplary method does not rely on epitope-MHC tetramers to identify the state of T cell activation in an allergen specific manner. In the exemplary method, allergen-specificity is determined by selective up-regulation of the CD154 molecule (CD40 ligand) following a brief stimulation with an allergen of interest. The desired allergen-specific activated T cells (e.g., pro-allergic T cells) can then be identified and/or recovered using a differentiation marker, such as CD27, CCR7, CD4SRB, or CD7. As described below, this dual screen for activation and differentiation markers, independent of epitope binding (e.g., not using a peptide epitope/MHCII tetramer), successfully identifies activated allergen-specific T_{H}2 subpopulations that are pathogenic and indicative of allergen sensitivity in the subject. Accordingly, the disclosed screen is useful for methods including:
detecting and quantifying pro-allergic (i.e., pathogenic) allergen-specific T cells;
isolating and purifying live pro-allergic allergen-specific T cells;
assessing the clinical status (e.g., “Allergic” vs. “Non-allergic”) of the patient;
determining achievement of clinical benefit in allergic patient receiving immunotherapy (e.g., monitoring immunotherapy); and
testing effect of drug compound on live pro-allergic allergen-specific T cells.

### Background

Currently, allergen-specific immunotherapy (ASIT) is the only known disease-modifying treatment of allergic diseases. The principle of ASIT is to administer gradually increasing doses of allergen, either as an allergen extract or as recombinant allergen. While evidence to date has revealed that ASIT can be clinically efficacious, a long period of time may elapse before achievement of clinical benefit and a subset of patients appears to be clinically unresponsive to ASIT. In the absence of information about primary clinical endpoints, biomarkers can provide critical insights that allow investigators to guide the clinical development of the candidate vaccine. However, assumptions about a correlation between immunological end-points and clinical outcomes of allergy vaccine are not supported by current monitoring strategies. Given their pivotal role in both the induction and control of allergic inflammation, quantifiable changes at the level of CD4+ T cells could represent a clinically meaningful signature that will reflect and quantify an underlying allergic disease process. This, in turn, would facilitate the monitoring of clinical outcomes, facilitate design and evaluation of allergy vaccines, and enable our understanding of mechanisms of action associated with successful immunotherapy.

The recent use of antigen-specific T cell assay in the allergy context demonstrated that the degree of differentiation of pollen allergen-specific CD4+ memory T cells is correlated with their functional activities and sensitivity to ASIT. These data have several important implications for understanding the basic immunologic mechanisms involved in the amelioration of allergic symptoms during allergen-SIT. First, it demonstrates that allergic and non-allergic individuals have functionally and phenotypically distinct circulating subsets of allergen-specific CD4+ T cells, which can be clearly differentiated based on their differentiation stage (e.g., as indicated by CD27 expression status). Both of these subsets actively respond to natural allergen exposure, but they appear to play markedly different roles in allergic disease. Specifically, while CD27+ allergen-specific memory CD4+ T cells are detected in both allergic and non-allergic subjects, CD27− allergen-specific memory CD4+ T cells are exclusively observed in allergic subjects. In contrast to CD27+ allergen-specific CD4+ T cells, CD27− allergen-specific CD4+ T cells are confined to allergic subjects and associated with T_{H}2 cytokine production, providing a clear functional connection with allergic disease. Finally, successful ASIT leads to selective elimination of CD27− allergen-specific T cells in the peripheral blood without significant changes in the CD27+ counterpart. These data suggest a novel mechanism in which the depletion of CD27− allergen-specific T cells might be a prerequisite for the induction of specific tolerance. Importantly, this could lead to the development of predictive markers for the clinical success of ASIT but also to new vaccine strategies to enhance the power of allergen-specific immunotherapy. In this context, monitoring CD27 expression on antigen-specific T cells provide a valuable tool for assessing the clinical status (Allergic vs. Non-allergic) of the patient to a particular allergen and for determining achievement of clinical benefit in patient receiving allergen immunotherapy.

### Study Overview

To establish a reliable method to ascertain the presence of pathogenic T cells specific for an allergen, a protocol was developed that incorporated a screen for an activation marker (CD154) and a differentiation marker (CD27), each of which is independent of the functional immune receptors that bind to the allergen itself. The protocol was used applied to characterize the subpopulations of allergen-specific T cells from individuals that were allergic or not allergic to an allergen, as well as monitor the efficacy of immunotherapy in subjects that were allergic to an allergen.

### Screen Protocol

1. PBMCs were isolated from whole blood using density gradient centrifugation.

2. PBMCs (1-2×10⁷ cells) were then stimulated for 1 to 24 hrs in RPMI 1640 supplemented with 5% AB serum, with the antigen (e.g., allergen-derived peptides, protein, or allergen crude extract) in the presence of functional grade CD40 at 37° C. and 5% CO₂.

3. After stimulation, cells were centrifuged at 1200 RPM for 5 minutes.

4. Enriching antibodies (α-CD154) were added to cells at a volume of 3 μper 10 million cells.

5. Cells were vortexed and resuspended and allowed to incubate for 15-20 minutes at RT in the dark.

6. Cells were washed with 4 ml of PBS and centrifuged at 1200 RPM for 5 minutes and decanted.

7. 1 μl of Miltenyi anti-PE microbeads were added to each tube per million cells. Cells were then resuspended and vortexed, and allowed to incubate for 10-15 minutes at RT in the dark.

8. Cells were washed with 4 ml of PBS and centrifuged at 1200 RPM for 5 minutes and decanted.

9. During step 6, 1 ml of PBS was added to the appropriate number of Miltenyi MS columns loaded onto a magnet. Flow through was collected in a new falcon tube.

10. Cells were resuspended in 1 ml of PBS per 50 million cells. Resuspended cells were added to a Miltenyi MS column and allowed to completely pass through the column while the flow through was collected in the same falcon tube as step 7.

11. Another 1 ml of PBS was added to the MS column and three drops were allowed to fall into the flow through tube while still attached to the magnet. After three drops, the column was transferred to a new falcon tube and the remaining volume of PBS was allowed to flow through the column.

12. After the column was eluted, 1 ml of PBS was added to the liberated column and plunged at a rate of 2 drops/second into the elution falcon tube.

13. Elution column was centrifuged at 1200 RPM for 5 minutes and decanted.

14. Surface staining antibody cocktail (including at minimum CD154, CD4, CD45RO, and CD27) was then added to remaining volume of cells and allowed to incubate for 15-20 minutes at RT in the dark. Surface panel may include CRT_{H}2.

15. Cells were resuspended in 200 μl of PBS and analyzed via flow cytometry.

The described protocol can be applied to cells in a variety of sample formats, including running the protocol directly on whole blood instead of on isolated PBMCs. Furthermore, samples can be frozen prior to the protocol.

Allergen challenge is not limited to any particular allergen or format. The allergen can be presented in purified form, in crude extract, as whole allergen, as allergen fragment, or as epitope in MEW. Co-stimulation can be performed with any known stimulant as an alternative to CD40, or in addition to CD40, including but not limited to CD154, CD69, CD137, antigen/tetramer, and the like.

Enrichment or isolation of the activated allergen-specific T cells from the PBMCs, e.g., starting at step 4, above, is optional but not required for the ultimate detection and quantification of terminally differentiated activated allergen-specific T cells.

### Results

The above protocol was used to detect and quantify activated allergen-specific T cells from various subjects (e.g., allergic, non-allergic, or in various stages of treatment) to associate the presence or amount of various differentiation states with the allergic condition.

In a first series of assays, PBMCs were isolated from subjects who were previously identified as allergic or non-allergic to peanuts. The isolated PBMCs were stimulated for 2 to 24 hours of peanut crude extract (Stallergenes-Greer, Cambridge, Mass.) in the presence of at least 0.1 μg/ml anti-CD40 blocking antibody (Myltenyi Biotec, Cambridge, Mass.). After enrichment for CD154 using PE-conjugated α-CD154 antibodies and Miltenyi anti-PE microbeads, the cells were surface stained with various differentiation markers to identify various subsets of activated allergen specific T cells. After staining, the cells were analyzed using flow cytometry.

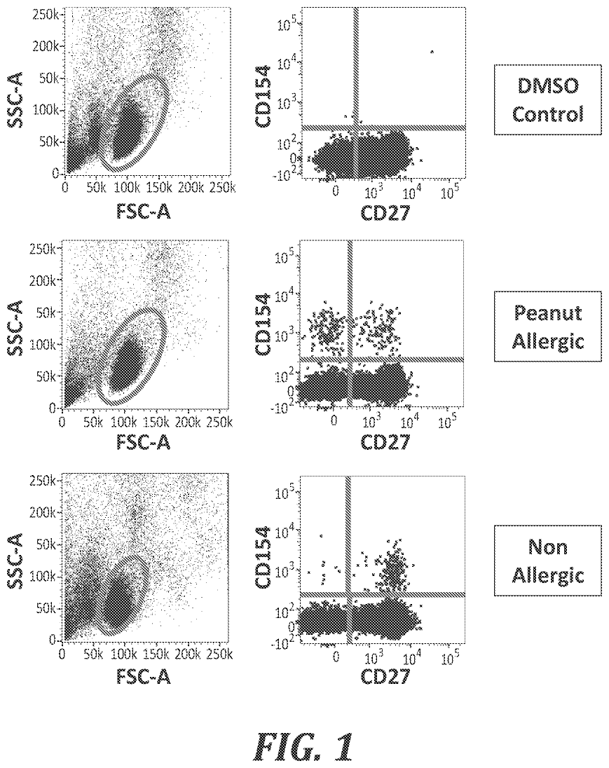

FIG. 1 illustrates flow cytometry analyses of cells obtained from peanut allergic or non-allergic individuals after the isolated cells were challenged with peanut allergen (or DMSO for control). The left panels illustrate the forward scatter and side scatter parameters of the cells with the lymphocytes indicated. DMSO-challenged cells show virtually no activated, allergen specific T cells (top row), whereas both experimental groups that were exposed to peanut allergen have subpopulations of CD154 expressing cells. These CD154 expressing cells from subjects with peanut allergies had subpopulations of CD27+ and CD27− cells, whereas the non-allergic subjects only had CD27+ cells.

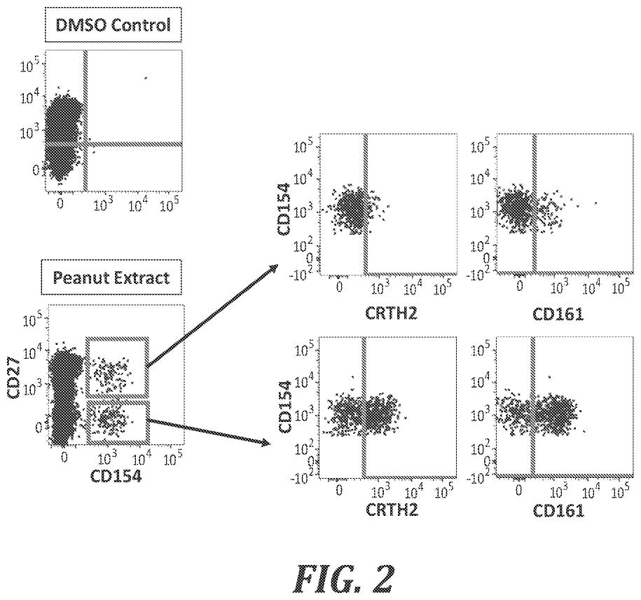

The two subpopulations of CD154+/CD27+ and CD154+/CD27− cells from subject with peanut allergy were further assessed for expression of CRT_{H}2 and CD161 to assess/confirm for pro-allergic properties. As illustrated in FIG. 2, only the CD154+/CD27− compartment included pro-allergic cells (CRTH2+ and CD161+), whereas the CD154+/CD27+ compartment did not.

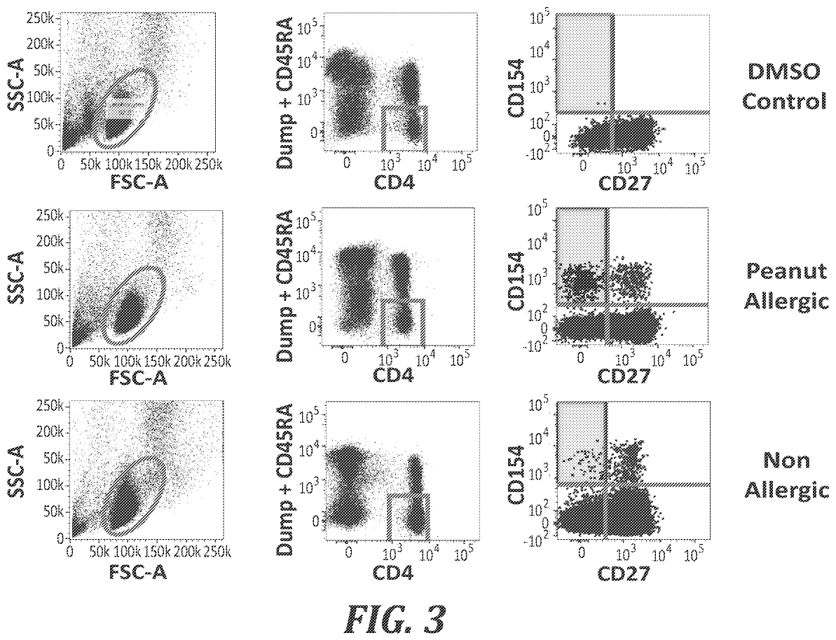

FIG. 3 illustrates additional flow cytometry characterization of peanut allergen-challenged PBMCs from subjects with and without peanut allergy. The left column shows the forward scatter and side scatter parameters of the cells with the lymphocytes indicated. The middle column shows the live memory (CD45RA−) CD4+ T cells from the lymphocytes population. The right column again demonstrates that activated allergen-specific T cells from subjects with peanut allergies contain a large subpopulation of CD27− cells, as well as a subpopulation of CD27− cells. In sharp contrast, the activated allergen-specific T cells from subjects with no peanut allergies have very few CD27− cells.

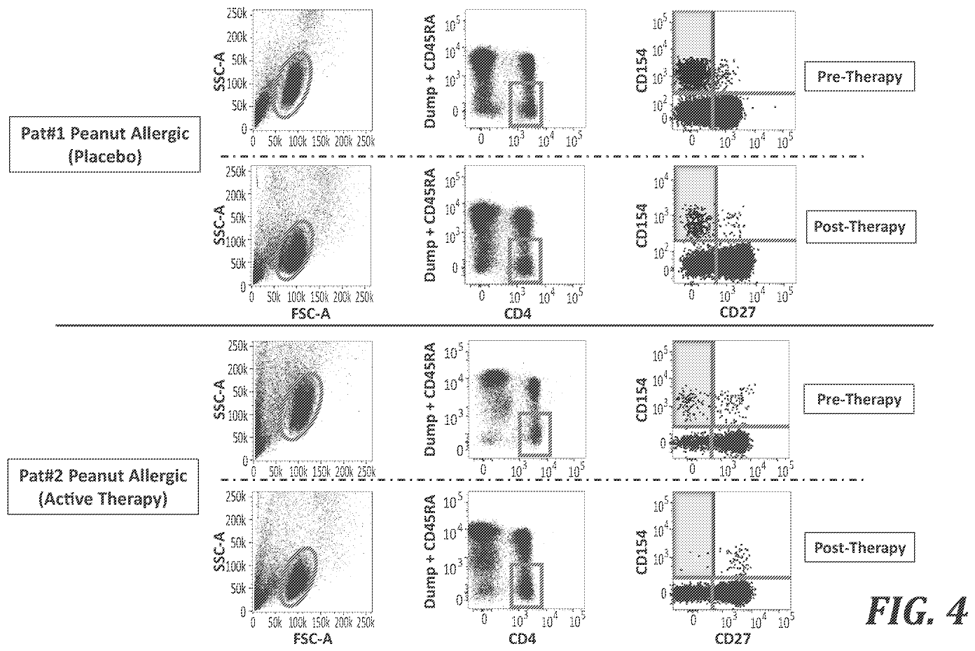

The above cell-profiling protocol was also applied to study the effects of immunotherapy on the subpopulations of activated T cells. Subjects with peanut allergies were divided into a placebo and immunotherapy groups. The placebo group received a mock immunotherapy regime using a peanut-tasting powder without allergen. The active immunotherapy group received allergen-specific immunotherapy (ASIT) that consisted of peanut flour (300 mg/day). PBMCs were obtained from all subjects prior to initiation of the placebo or active peanut ASIT, as well as after the termination of the placebo or active peanut ASIT. The PBMCs were challenged with a pool of peanut peptides library derived from Ara h 1, Ara h 2, Ara h 3, Ara h 6 and Ara h 8 peanut allergic components, and subjected to screening, as described above. FIG. 4 illustrates the flow cytometry analysis of the subjects' activated, allergen-specific T cell subpopulations. The left column shows the initial forward scatter and side scatter parameters of the cells with the lymphocytes indicated. The middle column shows the CD4+ cells live memory (CD45RA−) CD4+ T cells from the lymphocytes population. The right column shows the proportions of cells with CD154 expression and CD27 expression before and after therapy. Allergic subjects who were given allergen-specific immunotherapy (ASIT) using relevant peanut allergens experienced a significant decline in detectable activated peanut allergen-specific T cells that were also CD27−, indicated a significant decline in such terminally differentiated activated peanut allergen-specific T cells in subjects receiving appropriate immunotherapy for peanut allergies. This trend was not observed for allergic subjects receiving a mock immunotherapy. In these subjects, the placebo did not substantially reduce the proportion of activated peanut allergen-specific CD27− T cells, which remained the dominant subpopulation of the activated cells.

To establish that the above approach can be used as a platform approach to determine sensitivity of a subject to any allergen of choice and not just peanut allergies, additional assays were performed with a variety of unrelated allergens. PBMCs were isolated from subjects who were previously identified as allergic or non-allergic to a variety of unrelated allergens. The isolated PBMCs were stimulated for 2 to 24 hrs of crude allergen extracts (Stallergenes-Greer) in the presence of at least 0.1 μg/ml anti-CD40 blocking antibody (Myltenyi Biotec). After enrichment for CD154+ cells using PE-conjugated α-CD154 antibodies and Miltenyi anti-PE microbeads, the cells were surface stained with various differentiation markers to identify various subsets of activated allergen specific T cells. After staining, the cells were analyzed using flow cytometry.

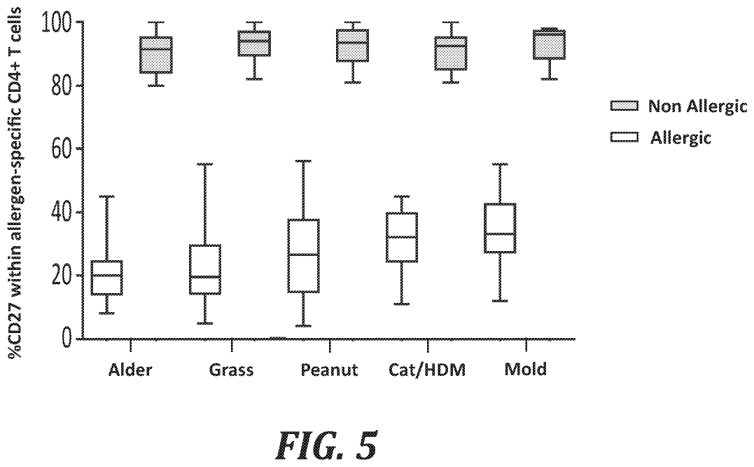

FIG. 5 is a graph illustrating the proportion of CD154+ T cells (activated allergen-specific CD4+ T cells) expressing CD27 for subjects with respect to five different allergens. These CD154 expressing cells from subjects with allergies had a lower proportion of activated allergen-specific cells expressing CD27+ (i.e., a higher proportion of activated allergen-specific cells that were CD27−) compared to the non-allergic subjects.

In another assay, PBMCs were isolated from subjects who were previously identified as allergic before and after receiving ASIT. The isolated PBMCs were stimulated for 2 to 24 hrs of various crude allergen extract (Stallergenes-Greer) in the presence of at least 0.1 μg/ml anti-CD40 blocking antibody (Myltenyi Biotec). After enrichment for CD154 using PE-conjugated α-CD154 antibodies and Miltenyi anti-PE microbeads, the cells were surface stained with various differentiation markers to identify various subsets of activated allergen specific T cells. After staining, the cells were analyzed using flow cytometry.

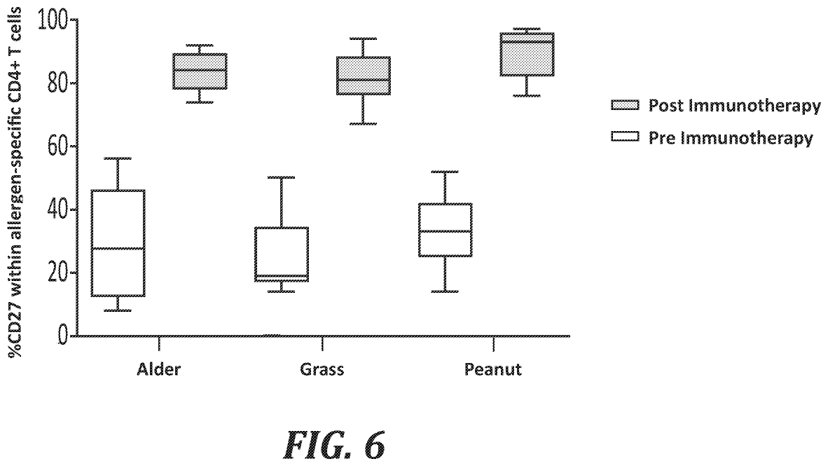

FIG. 6 is a graph illustrating the proportion of CD154+ (allergen-specific CD4+ T cells) expressing CD27 for subjects with respect to three different allergens, pre- and post-ASIT. Before ASIT all allergic subjects had low proportion of CD154+ (allergen-specific CD4+ T cells) expressing CD27. ASIT for all three allergens led to a dramatic increase of the proportion of CD154+ (allergen-specific CD4+ T cells) expressing CD27 (i.e., a dramatic decrease in activated allergen-specific cells that were CD27−).

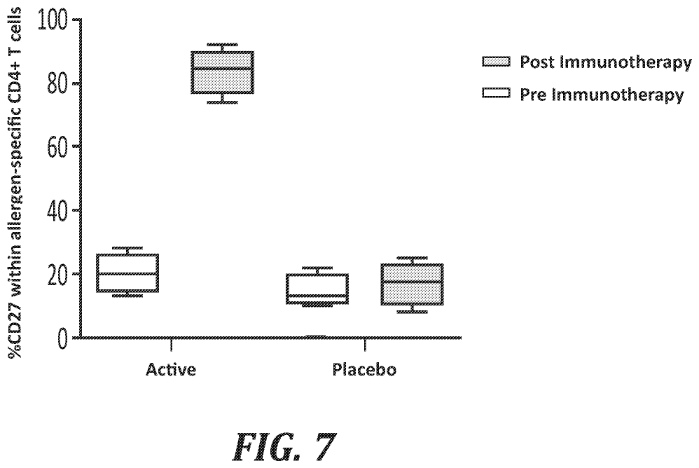

FIG. 7 is a graph illustrating change in the CD154+ T cells (allergen-specific T cells) expressing CD27 for peanut allergic subjects undergoing ASIT or Placebo during a clinical trial.

Finally, in another assay PBMCs were isolated from peanut allergic subjects who were previously identified as allergic before and after ASIT or similar subjects receiving placebo control treatment. As before, the isolated PBMCs were stimulated for 2 to 24 hours with a pool of peanut peptides library derived from Ara h 1, Ara h 2, Ara h 3, Ara h 6 and Ara h 8 peanut allergic components, in the presence of at least 0.1 μg/ml anti-CD40 blocking antibody (Myltenyi Biotec). After enrichment for CD154 using PE-conjugated α-CD154 antibodies and Miltenyi anti-PE microbeads, the cells were surface stained with various differentiation markers to identify various subsets of activated allergen specific T cells. After staining, the cells were analyzed using flow cytometry.

As illustrated in FIG. 7, before either therapy (ASIT or Placebo) all peanut allergic subjects had low proportion of CD154+ (allergen-specific CD4+ T cells) expressing CD27 (i.e., a high proportion of allergen-specific CD4+ T cells that are CD27−). ASIT led to a dramatic increase of the proportion of CD154+ (allergen-specific CD4+ T cells) expressing CD27 (i.e., a drastic decrease in the proportion of CD154+/CD27− cells) and this correlated with clinical benefit. In contrast, no significant changes were observed in the placebo group.

### Conclusion

These results demonstrate that the dual screen for an activation marker (e.g., CD154) and for differentiation (e.g., CD27) can accurately detect activated allergen-specific T cells that are pathogenic and cause sensitivity of the subject to the allergen in question. Specifically, the cells expressing CD154 (CD154+) but concurrently lack CD27 expression (CD27−) are shown to be pathogenic activated allergen-specific T cells and, thus, their presence indicates sensitivity of the subject to the allergen. Subjects receiving immunotherapy for the allergen demonstrate a marked reduction in CD154+/CD27− cells (and conversely, a marked increase in CD154+/CD27+ cells), which is associated with a simultaneous reduction in sensitivity. The absence of CD27 expression in activated allergen-specific T cells is an indicator of terminal differentiation of these cells. This indicates a mechanistic role of terminally differentiated activated allergen-specific T cells in allergies, which can be used as a target to detect sensitivity to allergens and monitor therapies thereto. As demonstrated here, the disclosed screen can monitor the presence of such pathogenic terminally differentiated and can facilitate monitoring of their reduced presence during treatment as an indicator of the efficacy of the treatment.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of specifically labeling a subpopulation of activated allergen-specific pathogenic T cells, comprising:
contacting a cell population comprising T cells with a suspected allergen to provide a challenged cell population,
contacting the challenged cell population with a first molecule that specifically binds to an activation marker for an activated allergen-specific T cell, wherein binding of the first molecule to the marker on a cell indicates that the cell is an activated allergen-specific T cell, and
contacting the challenged cell population, or a subpopulation thereof comprising an activated allergen-specific T cell, with a second molecule that specifically binds to a marker for a state of differentiation of the activated allergen-specific T cells.

2. The method of claim 1, further comprising determining the presence of a subpopulation of activated allergen-specific T cells that is indicated to be in a terminally differentiated state.

3. The method of claim 2, wherein determining the presence of activated allergen-specific T cells comprises detecting binding of the first molecule to the activation marker for T cell allergen-specificity within the challenged cell population, or the subpopulation thereof.

4. The method of claim 2 or claim 3, wherein determining the terminally differentiated state of a subpopulation of the activated allergen-specific T cells comprises determining a binding status of the second molecule to the marker for a state of differentiation.

5. The method of claim 4, further comprising quantifying the proportion of activated allergen-specific T cells specifically bound by the second molecule to the activated allergen-specific T cells not specifically bound by the second molecule to provide the binding status.

6. The method of claim 1 or claim 2, further comprising enriching for the activated allergen-specific T cells.

7. The method of claim 1, further comprising isolating subpopulation of the activated allergen-specific T cells in a state of terminal differentiation.

8. The method of claim 6 or claim 7, wherein isolating comprises use of flow cytometry or magnetic beads.

9. The method of claim 1, wherein the cell population comprises peripheral blood mononuclear cells (PBMCs) obtained from a subject.

10. The method of claim 9, further comprising obtaining the PBMCs from the subject.

11. The method of claim 1, wherein the activation marker for antigen specificity is selected from CD154, CD137, CD69, OX40 CD71, and CD25.

12. The method of claim 11, wherein the activation marker for an activated allergen-specific T cell is CD154.

13. The method of claim 11, wherein the activation marker for an activated allergen-specific T cell is CD69.

14. The method of one of claims 1, 2, and 11-13 wherein the marker for a state of differentiation of activated allergen-specific T cells is selected from CD27, CD45RB, CCR7, CRT_{H}2, CCR8, CD7, CD49b, CD49d, CD161, ST2, IL17RB, HPGDS, and CD200R.

15. The method of claim 14, wherein the expression of CRT_{H}2 (CRT_{H}2+), CCR8 (CCR8+), CD49b (CD49b+), CD49d (CD49d+), ST2 (ST2+), IL17RB (IL17RB+), HPGDS (HPGDS+), CD200R (CD200R+), or CD161 (CD161+) is indicative of a terminally differentiated state of the activated allergen-specific T cells.

16. The method of one of claim 2, 7, or 14, wherein the diminished or lack of expression of CD27 (CD27−), CD45RB (CD45RB−), CD7 (CD7−), or CCR7 (CCR7−) is indicative of a terminally differentiated state of the activated allergen-specific T cells.

17. The method of claim 1, 2, or 16, wherein the marker for a terminally-differentiated activated allergen-specific T cell is CD27, wherein the diminished or lack of expression of CD27 (CD27−) is indicative of a terminally differentiated state of the activated allergen-specific T cells.

18. The method of claim 15, wherein a determined presence of at least one marker indicating terminal differentiation of the one or more activated allergen-specific T cells is indicative that the subject is allergic with respect to the suspected allergen.

19. The method of claim 16, wherein a determined lack of at least one marker indicating terminal differentiation of the one or more activated allergen-specific T cells is indicative that the subject is allergic with respect to the suspected allergen.

20. The method of claim 1, wherein the first molecule or second molecule is an antibody, antibody-like molecule, receptor, aptamer, or a functional antigen-binding fragment or domain thereof.

21. The method of claim 20, wherein the antibody-like molecule is a single-chain antibody, a bispecific antibody, a Fab fragment, or a F(ab)₂ fragment.

22. The method of claim 21, wherein the single-chain antibody is a single chain variable fragment (scFv), single-chain Fab fragment (scFab), V_{H}H fragment, V_{NAR}, or nanobody.

23. The method of any one of claims 1-22, wherein detection of binding of the first molecule to the activation marker and/or binding of the second molecule to the marker for the state of differentiation comprises use of Fluorescence-activated cell sorting (FACS) or mass cytometry (CyTOF).

24. A method of determining whether a subject is allergic to a suspected allergen, comprising:
contacting whole blood or peripheral blood mononuclear cells (PBMCs) obtained from the subject with the suspected allergen to provide challenged PBMCs,
contacting the challenged PBMCs with a first molecule that specifically binds to an activation marker for an activated allergen-specific T cell, wherein binding of the first molecule to the marker on a cell indicates that cell is an activated allergen-specific T cell,
contacting the challenged PBMCs or activated allergen-specific T cells determined therefrom with a second molecule that specifically binds to a marker for a state of differentiation of activated allergen-specific T cells, and
determining the presence of a subpopulation of activated allergen-specific T cells that are indicated to be in a terminally differentiated state, wherein the presence of an activated allergen-specific T cell subpopulation that is terminally differentiated indicates that the subject is allergic to the allergen and the absence of an activated allergen-specific T cell subpopulation that is terminally differentiated indicates that the subject is not allergic to the allergen.

25. The method of claim 24, further comprising treating the subject's allergic condition.

26. The method of claim 24, wherein treating the subject comprises administering immunotherapy.

27. A method of monitoring the presence of activated allergen-specific T cells in a subject allergic to the allergen, the method comprising performing the following with peripheral blood mononuclear cells (PBMCs) obtained from the subject at two or more time points:
contacting the PBMCs with the allergen to provide challenged PBMCs,
contacting the challenged PBMCs with a first molecule that specifically binds to an activation marker for an activated allergen-specific cell, wherein binding of the first molecule to the marker on a cell indicates that the cell is an activated allergen-specific T cell,
contacting the challenged PBMCs or activated allergen-specific T cells determined therefrom with a second molecule that specifically binds to a marker for a state of differentiation of activated allergen-specific T cells, and
determining the relative abundance over time of a subpopulation of activated allergen-specific T cells that are indicated to be in a terminally differentiated state, wherein a decreased abundance of terminally differentiated activated allergen-specific T cells indicates that the subject is becoming less allergic to the allergen.

28. The method of claim 27, wherein at least one of the two or more time points occurs during or after treatment for the subject's allergic condition.

29. The method of claim 27 or claim 28, wherein the eventual absence of terminally differentiated activated allergen-specific T cells indicates that the subject is no longer allergic to the allergen.

30. A method of monitoring the efficacy of the immunotherapy of a subject that is allergic to an allergen, comprising performing the following with peripheral blood mononuclear cells (PBMCs) obtained from the subject at one or more time points during immunotherapy:
contacting the PBMCs with the allergen to provide challenged PBMCs,
contacting the challenged PBMCs with a first molecule that specifically binds to an activation marker for an activated allergen-specific T cell, wherein binding of the first molecule to the marker on a cell indicates that cell is an activated allergen-specific T cell,
contacting the challenged PBMCs or activated allergen-specific T cells determined therefrom with a second molecule that specifically binds to a marker for a state of differentiation of activated allergen-specific T cells, and
determining the relative abundance over time of a subpopulation of activated allergen-specific T cells that are indicated to be in a terminally differentiated state, wherein a decreased abundance of terminally differentiated activated allergen-specific T cells indicates that the efficacy of the immunotherapy.

31. The method of one of claim 24, 27, or 30, wherein determining the terminal differentiated state of a subpopulation of the activated allergen-specific T cells comprises determining a binding status of the second molecule to the marker for a state of differentiation.

32. The method of claim 31, further comprising quantifying the proportion of activated allergen-specific T cells specifically bound by the second molecule to the activated allergen-specific T cells not specifically bound by the second molecule to provide the binding status.

33. The method of one of claim 24, 27, or 30, further comprising enriching for the activated allergen-specific T cells.

34. The method of claim 24, further comprising isolating the subpopulation of the activated allergen-specific T cells in a state of terminal differentiation.

35. The method of claim 33 or 34, wherein enriching or isolating comprises use of flow cytometry or magnetic beads.

36. The method of one of claim 24, 27, or 30, wherein the method comprises obtaining the PBMCs from the subject.

37. The method of one of claim 24, 27, or 30, wherein the activation marker for allergen specificity is selected from CD154, CD137, CD69, OX40, CD71, and CD25.

38. The method of claim 37, wherein the activation marker for an activated allergen-specific T cell is CD154.

39. The method of claim 37, wherein the activation marker for an activated allergen-specific T cell is CD69.

40. The method of any one of claims 24, 27, 30, and 37-39, wherein the marker for a state of differentiation of activated allergen-specific T cells is selected from CD27, CD45RB, CCR7, CRT_{H}2, CCR8, CD7, CD49b, CD49d, CD161, ST2, IL17RB, HPGDS, and CD200R.

41. The method of claim 40, wherein the expression of CRT_{H}2 (CRT_{H}2+), CCR8 (CCR8+), CD49b (CD49b+), CD49d (CD49d+), ST2 (ST2+), IL17RB (IL17RB+), HPGDS (HPGDS+), CD200R (CD200R+), or CD161 (CD161+) is indicative of a terminally differentiated state of the activated allergen-specific T cells.

42. The method of claim 40, wherein the diminished or lack of expression of CD27 (CD27−), CD45RB (CD45RB−), CD7 (CD7−), or CCR7 (CCR7−) is indicative of a terminally differentiated state of the activated allergen-specific T cells.

43. The method of one of claims 24, 27, 30, 37-40, and 42, wherein the marker for a terminally-differentiated activated allergen-specific T cell is CD27, wherein the diminished or lack of expression of CD27 (CD27−) is indicative of a terminally differentiated state of the activated allergen-specific T cells.

44. A kit, comprising a first molecule that specifically binds to a marker for an activated allergen-specific T cell and a second molecule that specifically binds to a marker for a state of differentiation of activated allergen-specific T cells.

45. The kit of claim 44, wherein the first molecule or second molecule is an antibody, antibody-like molecule, receptor, aptamer, or a functional antigen-binding fragment or domain thereof.

46. The kit of claim 45, wherein the antibody-like molecule is a single-chain antibody, a bispecific antibody, a Fab fragment, or a F(ab)₂ fragment.

47. The kit of claim 46, wherein the single-chain antibody is a single chain variable fragment (scFv), single-chain Fab fragment (scFab), V_{H}H fragment, V_{NAR}, or nanobody.

48. The kit of claim 44, wherein the first molecule specifically binds to CD154 and the second molecule specifically binds to CD27.

49. The kit of claim 44, wherein the first molecule specifically binds to CD69 and the second molecule specifically binds to CD27.
